(19) United States
(12) Patent Application Publication
(10) Pub. No.: US 2024/0238059 A1
(43) Pub. Date: Jul. 18, 2024

(54) Grip with Disposable Barrier

(71) Applicant: FK Irons Inc., Doral, FL (US)

(72) Inventors: Gaston Alfonso Siciliano, Bal Harbour, FL (US); Christian Joseph Bonomo, Homestead, FL (US); Guillermo Gabriel Paz Estevez, Homestead, FL (US)

(73) Assignee: FK Irons Inc., Doral, FL (US)

(21) Appl. No.: 18/411,312

(22) Filed: Jan. 12, 2024

Related U.S. Application Data
(60) Provisional application No. 63/438,706, filed on Jan. 12, 2023.
(60) Provisional application No. 63/470,130, filed on May 31, 2023.
(60) Provisional application No. 63/599,321, filed on Nov. 15, 2023.

(51) Int. Cl. A61B 46/10; A61M 37/00

(52) U.S. Cl. CPC A61B 46/10; A61M 37/0076; A61M 2205/586

(57) ABSTRACT

A disposable barrier for use with a grip of a tattoo machine is disclosed. The barrier may be utilized to prevent contamination of tattoo machine components and to facilitate sanitary use of the tattoo machine. The barrier may include a securing portion that is utilized to secure the barrier to a portion of the grip. The barrier may also include a barrier bag portion attached to the securing portion and which is configured to extend away from the securing portion to cover a portion of the grip, the tattoo machine secured to the grip, any other componentry, or a combination thereof. The barrier bag portion may be collapsed towards the securing portion, such as when securing the tattoo machine to the grip. Once the barrier is utilized during a tattoo session the barrier may be discarded and a replacement disposable barrier may be secured to the grip for future use.

### CROSS-REFERENCE TO RELATED APPLICATIONS

This application claims priority benefit to U.S. Provisional Application No. 63/438,706, filed Jan. 12, 2023, U.S. Provisional Application No. 63/470,130, filed May 31, 2023 and U.S. Provisional Application No. 63/599,321, filed Nov. 15, 2023, the entireties of which are incorporated herein by reference.

### FIELD OF THE INVENTION

The present application relates to tattoo machines, grips for tattoo machines, protective barriers, anti-contamination accessories, tattoo accessories, and, more particularly, to a grip featuring a disposable barrier for use with a tattoo machine.

### BACKGROUND

Tattooing continues to be increasingly popular and the demand for high-quality tattoo artists and high-quality tattoo designs has similarly grown. As individuals seek out highly skilled tattoo artists, such individuals are also increasingly requesting higher standards of cleanliness and more robust sanitation protocols in order to reduced risk of potential infection and/or contamination of tattoo machine equipment and related accessories. This has been especially true in the context of recent pervasive pandemics where cleanliness and sterilization of tattoo machines and accompanying accessories has risen to higher levels of importance to consumers. In particular, consumers want to be confident that the tattoo machine utilized to apply a tattoo on a previous customer is devoid of contamination prior to being used to apply a tattoo on them. Based on at least the foregoing, consumers are actively searching for tattoo artists that can not only apply high-quality and highly detailed tattoos, but that can also do so in a safe, sanitary, and effective manner.

In order to placate consumers, tattoo artists and tattoo establishment proprietors have spent significant resources in training tattoo artists and developing tattoo machines, sanitation technologies, tattoo-machine accessories, and other related technologies and products. Additionally, tattoo establishments have instituted various types of protocols to reduce the potential for contamination, infection, and/or transmission of diseases. For example, tattoo establishments have instituted protocols that require tattoo artists to sterilize tattoo machine equipment and accessories after each use. As another example, tattoo establishments have installed sanitizer dispensers, anti-bacterial/anti-viral wet wipe dispensers, and the like to reduce potential transmission of various types of illnesses and/or infections. Despite the foregoing advancements and enhanced protocols, currently existing technologies still fail to reduce contamination and/or transmission of disease vectors at the levels sought by consumers and tattoo establishments.

Based on at least the foregoing, existing tattoo machine technologies may be enhanced to provide enhanced capabilities for preventing contamination of tattoo machines, enhancing cleanliness, and reducing the spread of disease vectors. Currently existing technologies often involve using sprays and wipes between tattoo machine users. While such techniques do help in reducing contamination, there is still room for improvement. Additionally, existing technologies often fail to protect the tattoo machine equipment itself and may allow tattoo ink to potentially damage tattoo machine components, particularly when ink flows into the internals of the tattoo machine. Based on at least the foregoing, current tattoo machine technologies may be improved and enhanced to provide for enhanced tattoo machine protection, enhanced customer satisfaction, increased flexibility, greater versatility, more robust sanitation protocols, and increased ease-of-use.

### SUMMARY

A disposable elastic membrane barrier for use with a grip of a tattoo machine is disclosed. In some arrangements, the tattoo machine can rotate freely and independently of the disposable elastic membrane barrier so that the elastic membrane barrier maintains the machine in a sterile and clean environment.

A disposable elastic membrane barrier for use with a grip of a tattoo machine is disclosed. The elastic membrane barrier may be utilized to prevent contamination of tattoo machine components and to facilitate sanitary use of the tattoo machine, such as during tattoo sessions being performed on different tattoo recipients. In certain embodiments, the elastic membrane barrier may include a securing portion that may be utilized to secure the elastic membrane barrier to a portion of the grip. The elastic membrane barrier may also include a barrier bag portion attached to the securing portion. The barrier bag portion may be configured to extend and/or roll away from the securing portion to cover a portion of the grip, a tattoo machine secured to the grip, any other componentry, or a combination thereof. In certain embodiments, the end of the barrier bag portion that is not secured to the securing portion may be sealed, such as by utilizing various types of seals, to prevent contamination of the tattoo machine from the top of the tattoo machine. After use of the tattoo machine or at other desired times, the barrier bag portion may be collapsed towards the securing portion, such as when securing the tattoo machine to the grip, removing the tattoo machine from the grip, or when inspecting the tattoo machine itself. Once the elastic membrane barrier is utilized during a tattoo session, the elastic membrane barrier may be discarded, and a replacement disposable barrier may be secured to the grip for a future tattooing session.

Notably, the disposable elastic membrane barrier may provide an ergonomic single use (or multiple use depending on the use-case scenario) component for preventing contamination of tattoo machine components, preventing ink from flowing into the tattoo machine, ensuring a sterile tattoo machine during use, and enhancing general tattoo machine and artist cleanliness. The clastic membrane barrier may be locked down onto the grip, glued onto the grip, connected to the grip, and/or otherwise secured to the grip depending on preference. The disposable elastic membrane may be made of materials, such as, but not limited to, rubber, silicone, plastics, and/or other materials to facilitate tattoo artist comfort when utilizing the tattoo machine during a tattooing session. In certain embodiments, the bag portion of the elastic membrane barrier may be configured to be spun around independently of the grip, which may thereby enable the grip to adjust freely while still protecting the tattoo machine. The disposable elastic membrane barrier may be utilized on pen-styled tattoo machines and/or other similarly shaped devices to reduce contamination of components and reduce risk of transmission of disease vectors.

In one embodiment, a disposable elastic membrane barrier for use with a grip of a tattoo machine is provided. The disposable elastic membrane barrier may include a securing portion configured to secure the disposable barrier to a portion of the grip. Additionally, the disposable elastic membrane barrier may include a barrier bag portion attached to the securing portion. In certain embodiments, the barrier bag portion may be configured to extend away from the securing portion to cover a portion of the grip, a tattoo machine secured to the grip, or a combination thereof, such as when the disposable elastic membrane barrier is in an open configuration. When the tattoo machine is no longer in use or at other desired times, the barrier bag portion of the disposable elastic membrane barrier may be collapsed towards the securing portion of the barrier, such as to place the barrier in a closed or undeployed configuration. After the tattoo machine is used with the disposable elastic membrane barrier, a user may remove the barrier from the grip and may secure a new barrier to the grip for a future use of the tattoo machine.

In another embodiment, a method for utilizing a disposable elastic membrane barrier is disclosed. In certain embodiments, the method may include providing the disposable elastic membrane barrier for the grip for use with a tattoo machine configured to be attached to the grip. Additionally, the method may include securing the disposable elastic membrane barrier to a portion of the grip. The method may proceed to include securing the tattoo machine to the grip. Once the tattoo machine is secured to the grip, the method may include pulling or otherwise extending a portion of the disposable elastic membrane barrier over the tattoo machine and associated componentry such that the disposable barrier covers at least a portion of the tattoo machine, the associated componentry, a portion of the grip, or a combination thereof. The method may then include utilizing the tattoo machine with the disposable elastic membrane barrier during a tattooing session with a tattoo recipient. Once the tattooing session is completed or at another desired time, the method may include removing the disposable elastic membrane barrier from the grip after the tattoo machine has been utilized. Moreover, the method may include securing a new disposable elastic membrane barrier to the portion of the grip prior to a next use of the tattoo machine.

According to yet another embodiment, a grip for use with a tattoo machine and accompanying disposable clastic membrane barrier is disclosed. In certain embodiments, the grip may include a portion configured to secure to a securing portion of the disposable elastic membrane barrier. The disposable elastic membrane barrier may also include a barrier bag portion attached to the securing portion of the disposable elastic membrane barrier. In certain embodiments, the barrier bag portion may be configured to extend away from the securing portion of the barrier to cover a portion of the grip, a tattoo machine secured to the grip, or a combination thereof. When the tattoo machine is not in use or at another desired time, the barrier bag portion of the disposable elastic membrane barrier may be collapsed towards the securing portion to uncover the portion of the grip, the tattoo machine secured to the grip, or a combination thereof. In certain embodiments, the disposable barrier may be removed, such as after tattoo machine use on a tattoo recipient, and a new disposable elastic membrane barrier may be secured to the grip for a next use of the tattoo machine.

These and other features of the disposable barrier and accompanying tattoo machine are described in the following detailed description, drawings, and appended claims.

### BRIEF DESCRIPTION OF THE DRAWINGS

FIG. 1A is a schematic diagram illustrating a grip with an accompanying disposable barrier according to an embodiment of the present disclosure.

FIG. 1B is a schematic diagram illustrating the disposable barrier of FIG. 1A in unrolled form according to an embodiment of the present disclosure.

FIG. 1C is a schematic diagram illustrating a cross-section view of the grip and accompanying disposable barrier of FIG. 1A according to an embodiment of the present disclosure.

FIG. 2A is a schematic diagram illustrating a grip with an accompanying disposable barrier according to another embodiment of the present disclosure.

FIG. 2B is a schematic diagram illustrating the disposable barrier of FIG. 2A in unrolled form according to another embodiment of the present disclosure.

FIG. 2C is a schematic diagram illustrating a cross-section view of the grip and accompanying disposable barrier of FIG. 2A according to an embodiment of the present disclosure.

FIG. 3A is a schematic diagram illustrating a grip with an accompanying disposable barrier according to a further embodiment of the present disclosure.

FIG. 3B is a schematic diagram illustrating the grip of FIG. 3A secured to a tattoo machine protected by the disposable barrier according to a further embodiment of the present disclosure.

FIG. 4A is a schematic diagram illustrating grip featuring a groove for securing with an accompanying disposable barrier according to a further embodiment of the present disclosure.

FIG. 4B is a schematic diagram illustrating the grip of FIG. 4A secured to the disposable barrier according to a further embodiment of the present disclosure.

FIG. 4C is a schematic diagram illustrating a tattoo machine being secured to a grip and which is protected by the disposable barrier according to a further embodiment of the present disclosure.

FIG. 4D is a schematic diagram illustrating the tattoo machine fully secured to the grip and which protected by the disposable barrier according to a further embodiment of the present disclosure.

FIG. 5 is a schematic diagram of a disposable barrier for use with a grip of a tattoo machine according to an embodiment of the present disclosure.

FIG. 6 is a schematic diagram of a disposable barrier for use with a grip of a tattoo machine according to another embodiment of the present disclosure.

FIG. 7 is a diagram illustrating a person utilizing a grip of a tattoo machine with a disposable barrier according to an embodiment of the present disclosure.

FIG. 8A is a schematic diagram illustrating a grip with an accompanying disposable barrier according to a further embodiment of the present disclosure.

FIG. 8B is a schematic diagram illustrating a grip with an accompanying disposable barrier that is unrolled according to a further embodiment of the present disclosure.

FIG. 9A is a schematic diagram of a grip assembly that includes a rotatable sleeve according to another embodiment of the present disclosure.

FIG. 9B illustrates an exploded view of the grip assembly of FIG. 9A according to an embodiment of the present disclosure.

FIG. 9C illustrates details of a core of the grip assembly of FIG. 9A according to an embodiment of the present disclosure.

FIG. 9D illustrates attachment of the sleeve to the core of the grip assembly of FIG. 9A according to an embodiment of the present disclosure.

FIG. 10 is a flow diagram illustrating a sample method for utilizing a grip with an accompanying disposable barrier according to an embodiment of the present disclosure.

FIG. 11 is a schematic diagram of a machine that may be utilized to facilitate the operation of a tattoo machine utilized with a grip and accompanying disposable barrier of the present disclosure.

FIG. 12A illustrates an exploded view of a grip assembly that includes a rotatable sleeve and/or rotatable grip according to another embodiment of the present disclosure.

FIG. 12B illustrates a cross section of the grip assembly of FIG. 12A showing the sleeve in an expanded state according to an embodiment of the present disclosure.

FIG. 12C illustrates a cross section of the grip assembly of FIG. 12A showing the sleeve in a scrunched, stored or non-deployed state according to an embodiment of the present disclosure.

FIG. 12D illustrates a perspective view of a grip assembly that includes a rotatable sleeve and/or rotatable grip according to another embodiment of the present disclosure.

FIG. 12E illustrates a perspective view of the grip assembly of FIG. 12D in a stored or undeployed state or arrangment.

FIG. 12F illustrates a top view of the grip assembly of FIG. 12D in a stored or undeployed state or arrangment.

FIG. 12G illustrates a top view of the grip assembly of FIG. 12D with the sleeve unraveled over a tatoo device.

### DETAILED DESCRIPTION OF THE DRAWINGS

A disposable elastic membrane barrier (e.g., barrier 103) for use with a grip (e.g., grip 102) of a tattoo machine (e.g., tattoo machine 120) is disclosed. The elastic membrane barrier may be utilized to prevent contamination of the tattoo machine and to simultaneously enhance sanitary use of the tattoo machine, such as during a tattoo session with a tattoo recipient. In certain embodiments, the elastic membrane barrier may include a securing portion that may be utilized to secure the elastic membrane barrier to a portion of the grip. The elastic membrane barrier may also include a barrier bag portion attached to or otherwise integrated with the securing portion. In certain embodiments, the barrier bag portion may be configured to extend and/or roll away from the securing portion to cover a portion of the grip, a tattoo machine secured to the grip, any other componentry, or a combination thereof. In certain embodiments, the end of the barrier bag portion that is not secured to the securing portion may be sealed, such as by utilizing a various types of seals, to prevent contamination of the tattoo machine via the top of the tattoo machine. After use, the barrier bag portion may be collapsed or contracted towards the securing portion, such as when securing the tattoo machine to the grip, removing the tattoo machine from the grip, replacing the disposable elastic membrane barrier, and/or when inspecting the tattoo machine. For example, once the elastic membrane barrier is utilized during a tattoo session, the elastic membrane barrier may be discarded, and a replacement disposable barrier may be secured to the grip for a future tattooing session.

Notably, the disposable elastic membrane barrier may provide an ergonomic single use (or multiple use depending on the use-case scenario) component for preventing contamination of tattoo machine components, potentially preventing ink from flowing into the tattoo machine, ensuring a sterile tattoo machine during use, and enhancing general tattoo machine and artist cleanliness. The elastic membrane barrier may be integrated onto the grip, glued onto the grip, connected to the grip, and/or otherwise secured to the grip depending on preference and/or embodiment. The disposable elastic membrane may be made of materials, such as, but not limited to, rubber, silicone, plastics, and/or other materials to facilitate tattoo artist comfort when utilizing the tattoo machine during a tattooing session. In certain embodiments, the bag portion of the elastic membrane barrier may be configured to be spun around independently of the grip, which may thereby enable the grip to adjust freely while still simultaneously protecting the tattoo machine. In certain embodiments, the disposable elastic membrane barrier may be utilized on pen-styled tattoo machines and/or other similarly shaped devices to reduce contamination of components and reduce risk of transmission of disease vectors.

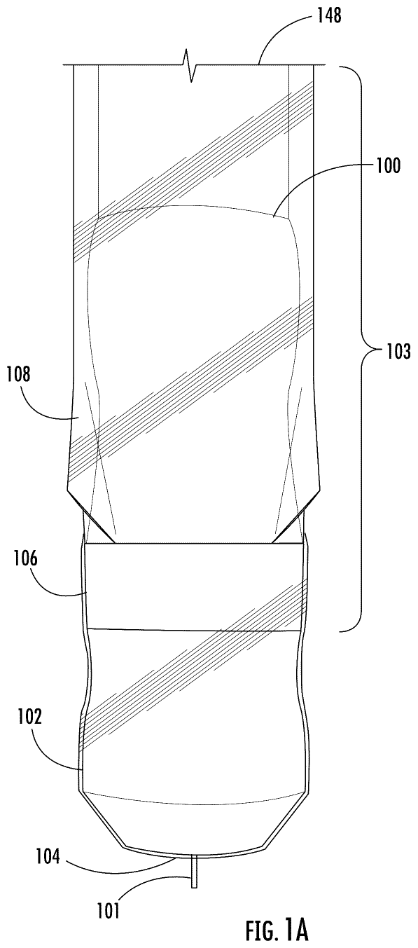

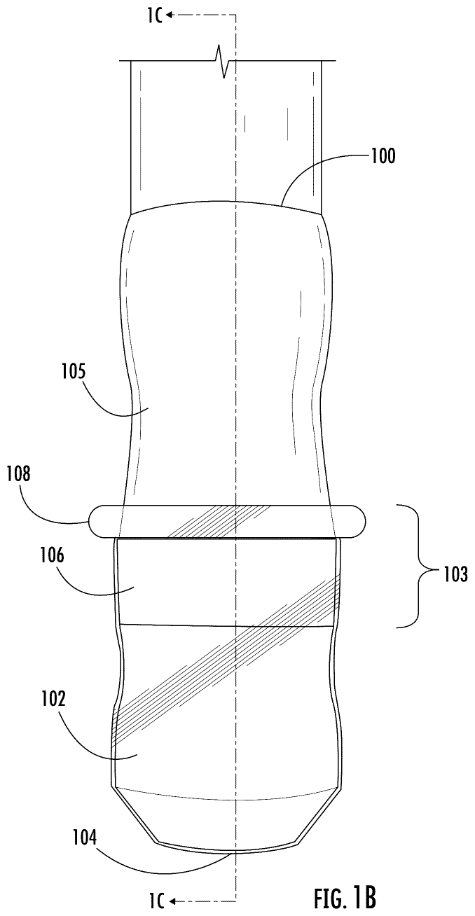

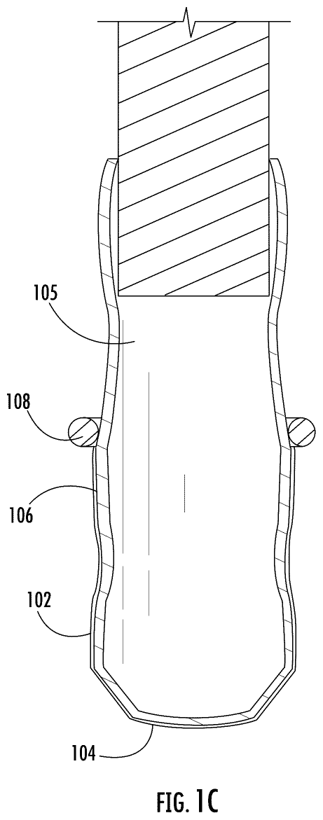

As shown in FIGS. 1A, 1B, 1C, 2A, 2B, 2C, 3A, 3B, 4A, 4B, 4C, 4D, 5, 6, 7, 8A, 8B, and 9A-9D, embodiments of disposable elastic membrane barriers for use with a grip (e.g., grip 102) of a tattoo machine (e.g., tattoo machine 120) are disclosed. The disposable elastic barriers 103 may be utilized to shield the componentry of a tattoo machine 120, such as while the tattoo machine 120 being utilized by a tattoo artist during a tattoo session with a tattoo recipient. The grip 102 may be utilized by a tattoo artist to hold onto the tattoo machine 120 and guide the tattoo machine 120 and needle 101 during a tattoo session. The tattoo machine 120 may be utilized to deliver ink to the skin (or other surface) of a user, such as during a tattoo session and/or procedure. The disposable elastic membrane barrier may serve as an accessory to the tattoo machine 115 and may be utilized to protect the tattoo machine 120 from contamination from disease vectors existing in the environment associated with the tattoo machine 120, bacteria, pathogens, and/or other contaminants. After each tattoo session or use, the disposable elastic membrane barrier may be removed and replaced with a new one to ensure tattoo machine 120 sterility and contamination free tattoo procedures for subsequent tattoo recipients. For example, if a disposable barrier is used during a first session with a first user, the tattoo artist may remove the disposable barrier after a tattoo session and replace the disposable barrier with a new disposable barrier for a tattoo session to be performed on a second user. Notably, the present disclosure provides for various embodiments of disposable barriers for use with grips 102 of tattoo machines 120, and depending on the circumstances, the tattoo artist may prefer one embodiment over the other.

Referring now specifically to FIGS. 1A, 1B, and 1C, an embodiment of a disposable elastic membrane barrier 103 for use with a grip 102 is shown. The grip 102 may be shaped and formed such that it is comfortable and ergonomic to hold in the hand of a tattoo artist. In certain embodiments, the grip 102 may be made of rubber, latex fabric, silicone, plastics (e.g., ABS plastic, polyethylene plastic, etc.), metals, or any combination thereof. In certain embodiments, the grip 102 may have an opening/receptacle 104 positioned at a top portion of the grip and another opening/receptacle 100 positioned at the bottom of the grip 102. The opening/receptacle 104 may be configured to receive a needle cartridge containing one or more needles 101 that may be utilized to transfer ink to the skin of a tattoo recipient when actuated and/or manipulated by the tattoo machine 120. The other opening/receptacle 100 may be configured to receive a tattoo machine 120 and may be configured to lock into place with the tattoo machine 120, have an interference fit with a portion of the tattoo machine 120, have a threaded portion that interfaces with a corresponding threaded portion on the tattoo machine 120 (e.g., screw fit), and/or affix to the tattoo machine 120 in any desired manner. In certain embodiments, the disposable elastic membrane barrier 103 may include a securing portion 106 and bag portion 108. The securing portion 106 may be the portion of the disposable elastic membrane barrier 103 that securely attaches the barrier 103 to the grip 102 of the tattoo machine 120, such as along a portion of the outer surface of the grip body 105 of the grip 102. In certain embodiments, the securing portion 106 may take any suitable form and utilize any suitable mechanism for securing the barrier 103 to the grip 102 and/or potentially to the tattoo machine 120. In FIGS. 1A, 1B, and 1C, the securing portion 106 may take the form of an clastic membrane (e.g., plastic or other suitable material) that may be adhered to a portion of the surface of the grip 102. In FIG. 1A, for example, the securing portion 106 has a generally cylindrical shape that is shaped such that it covers an out surface of a cylindrical portion of the grip 102 (other shapes and/or configurations may be utilized as well). The securing portion 106 may have adhesive on the inside surface of the securing portion 106 that allows the securing portion 106 to adhere to the grip 102. In certain embodiments, the securing portion 106 may be glued onto the portion of the grip 102. In certain embodiments, a heat gun may be utilized to warm a solution placed between the securing portion 106 and the portion of the grip 102 that the securing portion 106 is to be affixed to, and, upon cooling, the securing portion 106 may stick or otherwise adhere to the portion of the grip 102 when the solution cools.

As indicated herein, the disposable elastic membrane barrier 103 may also include a bag portion 108. The bag portion 108 may be made of similar materials as the securing portion 106, such as, but not limited to, plastic, rubber, elastic membrane material, stretchable materials, fabrics, any type of materials, or a combination thereof. In certain embodiments, the bag portion 108 may include a volume that may be configured to cover the tattoo machine 120 and/or other componentry that is affixed to the grip 102. In certain embodiments, such as when a tattoo machine 120 is not affixed to the grip 102 or is not in use, the bag portion 108 may be in a collapsed or compressed state, as is shown in FIG. 1B and the cross-section shown in FIG. 1C. In certain embodiments, the bag portion 108 may be configured to scrunch down towards the securing portion 108 (somewhat like an accordion or other similar mechanism), roll downwards (somewhat similar to a prophylactic), slide downwards, and/or otherwise collapse downwards to the securing portion 106. In the collapsed state, it may be easier to secure or remove the tattoo machine 120 to or from the receptacle 100 because the receptacle 100 may be exposed when the securing portion 108 is collapsed. When the tattoo machine 120 is going to be used or at other desired times, the bag portion 108 may be extended outwards and away from the securing portion 106 to cover an attached tattoo machine 120 and/or other componentry capable of being covered by the volume of the bag portion 108. When covering a tattoo machine 120 and/or other associated componentry, the bag portion 108 may reduce the possibility of contamination of the tattoo machine 120 and associated componentry. After the tattoo machine 120 has been used or at other designated times (e.g., the disposable clastic membrane barrier 103 is contaminated and/or is dirty), the disposable elastic membrane barrier 103 may be removed from the grip 102 and a new barrier 103 may be secured to the grip 102. A user may then cover the tattoo machine 120 and associated componentry with a new bag portion 108 and proceed with use of the tattoo machine 120 accordingly.

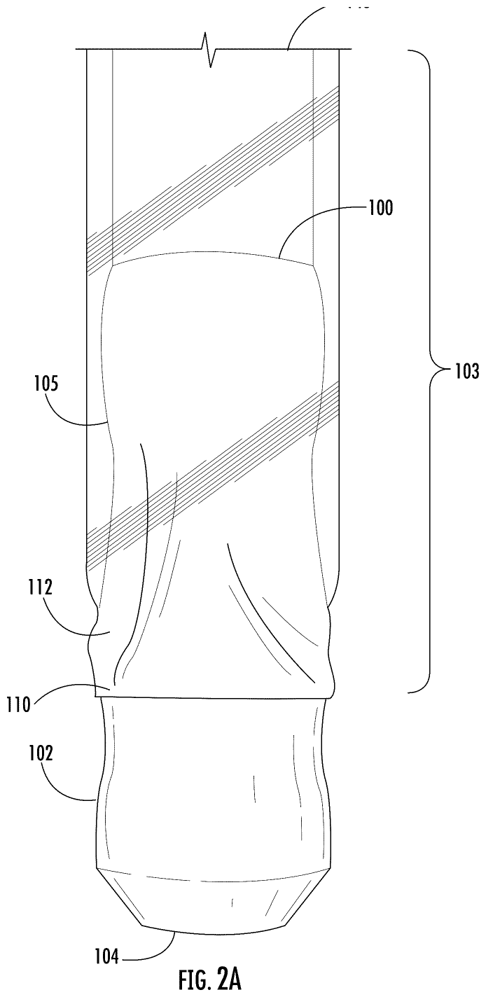

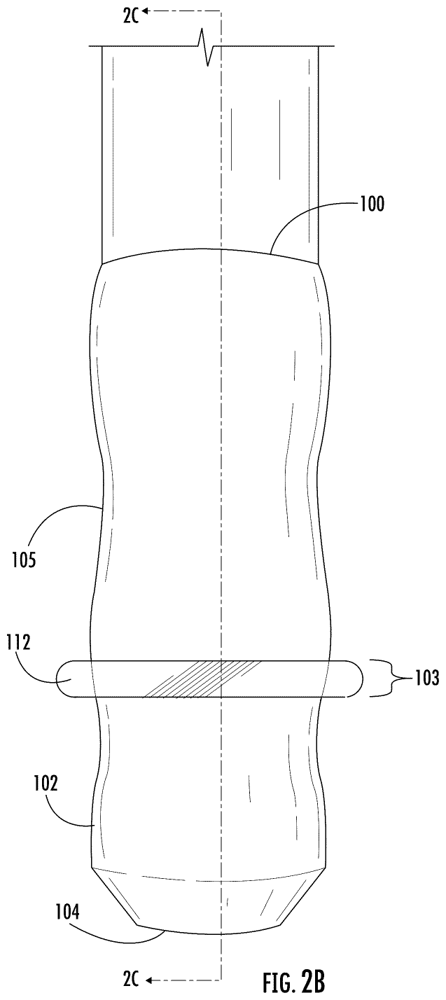

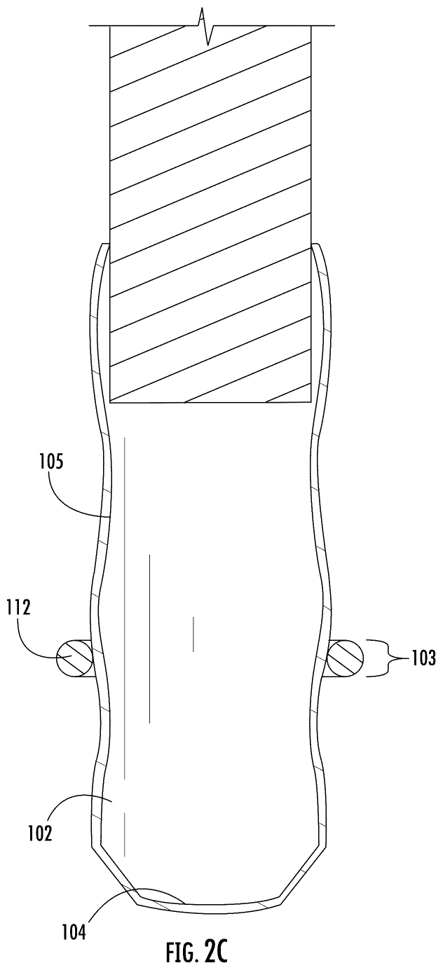

Referring now also to FIGS. 2A, 2B, and 2C, another embodiment of a disposable elastic membrane barrier 103 for use with a grip 102 is shown. Much like the embodiment shown in FIGS. 1A, 1B, and 1C, the embodiment of the disposable elastic membrane barrier 103 provided in FIGS. 2A, 2B, and 2C may include a securing portion 110 and a bag portion 112, which may be configured to cover a tattoo machine 120 attached to the grip and/or other componentry capable of being covered by the bag portion 112. In the embodiments provided in FIGS. 2A, 2B, and 2C, instead of having the securing portion 106 that adheres to the outside surface of a portion of the grip 102, the barrier 103 in this embodiment may be configured to secure within a pocket 109 of an overmolded portion 107 of the grip 102. For example, the securing portion 110 may have an adhesive on a surface of the securing portion 110 that may be configured to adhere within the pocket 109 of the overmolded portion 107 of the grip 102. In certain embodiments, a portion of the securing portion 110 may be taped within the pocket 109, secured directly onto an outer surface of the overmolded portion 107, or otherwise secured to the overmolded portion 107. Once the securing portion 110 is secured to the overmolded portion 107 and/or within the pocket 109, the bag portion 112 may be extended or rolled away from the securing portion 110 to cover a tattoo machine 120 attached to the grip 102. After use, the disposable barrier 103 may be replaced with a new disposable barrier 103.

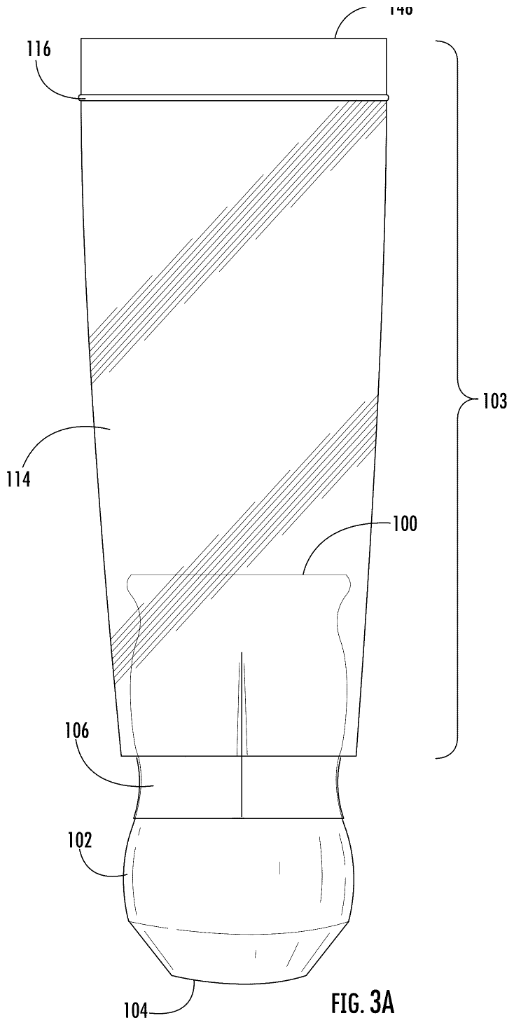

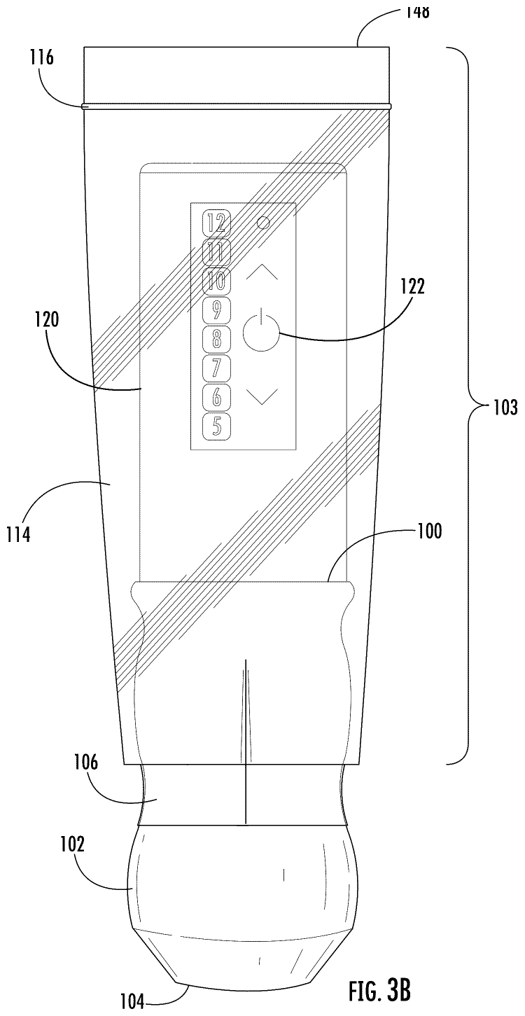

Referring now also to FIGS. 3A and 3B, another embodiment of a disposable clastic membrane barrier 103 for use with a grip 102 is shown. The disposable clastic membrane barrier 103 may include a securing portion 106 and a bag portion 114. The securing portion 106 may be much like the securing portion provided in FIGS. 1A, 1B, and 1C, and may be secured to an outer surface of the grip 102. For example, the securing portion 106 may be secured to the grip 102 via an adhesive applied to an inner surface of the securing portion 106, the securing portion may be secured to the grip 102 via tape, the securing portion may be secured to the grip 102 via use of a heat gun to warm up a solution positioned between the inner surface of the securing portion 106 and the outer surface of the grip 102, other securing mechanisms, or a combination thereof. Once the securing portion 106 is secured to the grip 102, the bag portion 114 may be extended away from the securing portion 106. The bag portion 114 may include a seal 116, which may be utilized to seal the bag portion 106, such as after the tattoo machine 120 is secured to the grip 102 and is covered with the bag portion 106. The seal 116 may be utilized to prevent contaminants from entering the top portion of the bag portion 114 and may be utilized to further enhance cleanliness routines and protocols. In certain embodiments, the seal 116 may be a zip-top, which is configured to seal as a user presses along the zip-top to seal the bag portion 114 closed. In the zip-top embodiment, the user may open the seal 116 by separating the zip-top. In certain embodiments, the seal 116 may include an adhesive to seal the top portion of the bag portion 114 closed, fasteners to seal the top portion of the bag portion 114, zippers, and/or other sealing technologies.

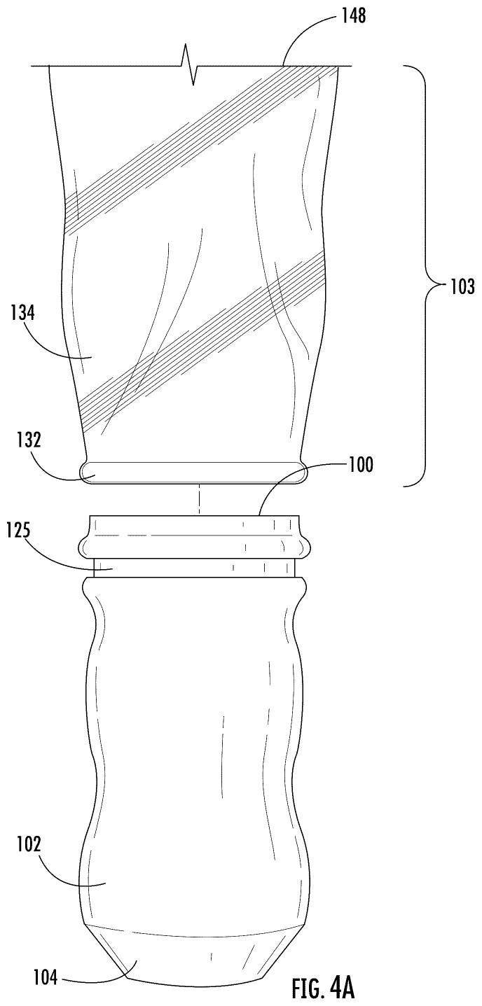

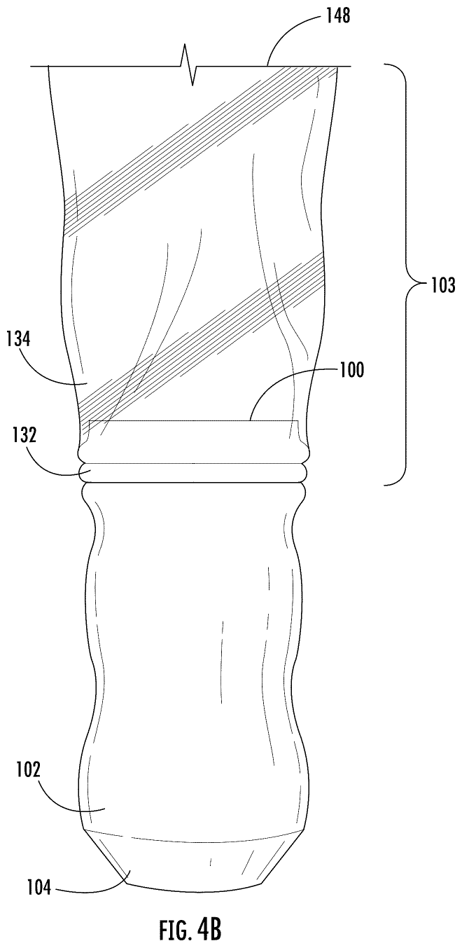

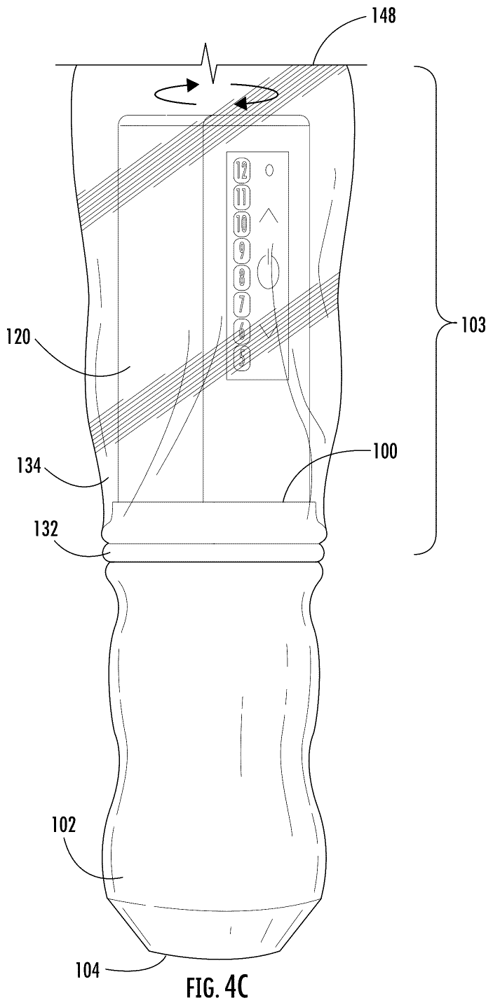

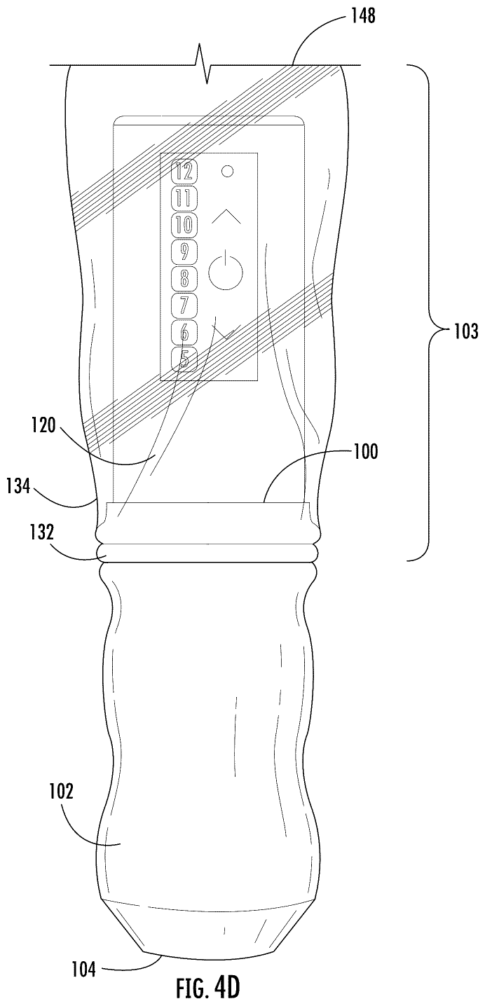

Referring now also to FIGS. 4A, 4B, 4C, and 4D, another embodiment of the disposable elastic membrane barrier 103 for use with a grip 102 of a tattoo machine 120 is shown. In certain embodiments, the disposable elastic membrane barrier 103 may include a securing portion 132 and a bag portion 134. The securing portion 132 may have a ring-shape has shown in FIGS. 4A, 4B, 4C, and 4D, which may be made of plastic, rubber, silicone, and/or other suitable materials. In certain embodiments, the securing portion 132 may have a size and shape such that the securing portion 132 may be configured to snugly fit and lock into place in the groove 125 of the grip 102. In certain embodiments, the grip 102 may be positioned down into the opening of the bag portion 134 until the securing portion 132 fits in to the groove 125 of the grip 102. When the securing portion 132 is secured within the groove 125, the disposable elastic membrane barrier 103 may be configured to spin around via the groove 125 and independently of the grip 102 and/or tattoo machine 120 connected to the grip 102 if the user of the tattoo machine 120 wishes to spin the barrier 103 based on the situation at hand. Alternativel, the grip 102 may be rotated independent of the disposable clastic membrane barrier 103 such that the barrier 103 remains stationary while the grip 102 is rotated. If the user wants to secure the tattoo machine 120 to the grip 102 while the barrier 103 is secured to the grip 102, the user can simply insert the tattoo machine 120 into the opening of the bag portion 134 and lock the tattoo machine 120 with the grip 102 by rotating a portion of the tattoo machine 120 into the receptacle 100 of the grip 102 until the tattoo machine 120 is locked into place with the grip, as shown in FIGS. 4C and 4D. In certain embodiments, the disposable elastic membrane barrier 103 may also include seal 116 depending on the preference of the tattoo artist and/or situation. When the disposable elastic membrane barrier 103 is used during a tattoo session by a tattoo artist with a tattoo recipient or at any other desired time, the artist may remove the barrier 103 from the grip and may install a new barrier 103 onto the groove 125 of the grip 102 so that the tattoo machine 120 is ready for a next use with a new tattoo recipient.

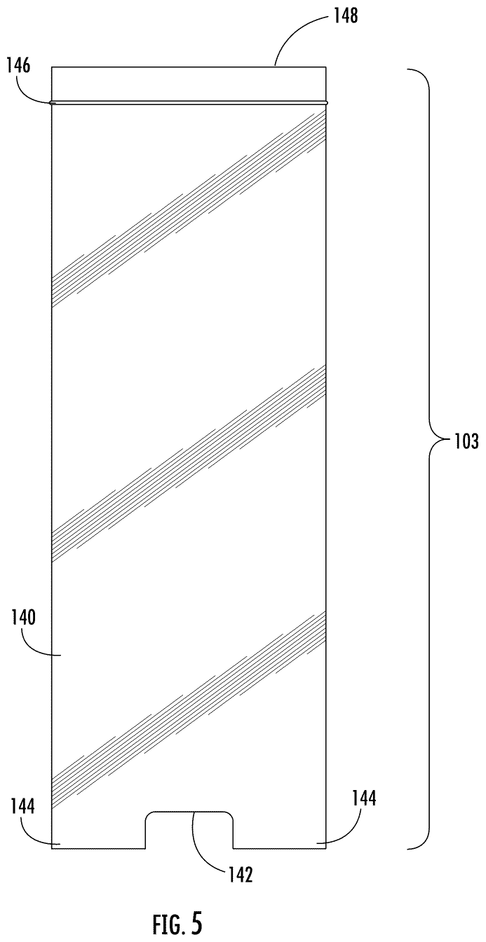

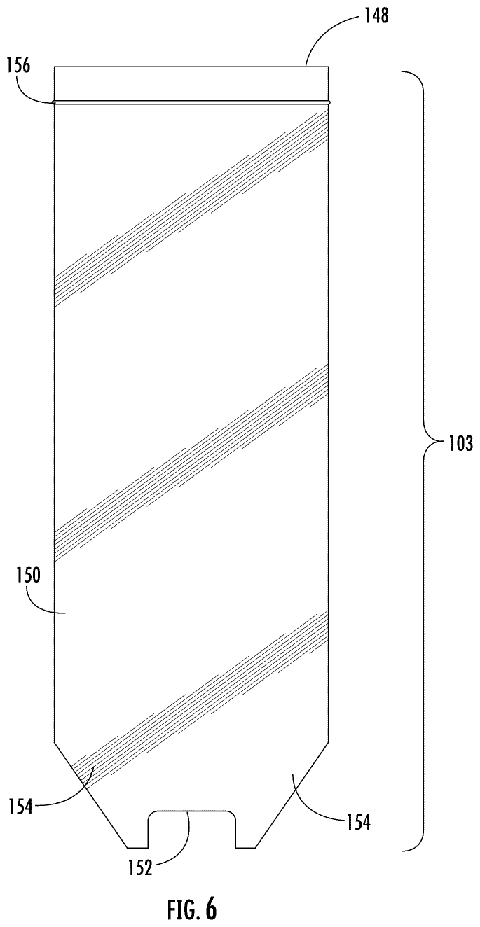

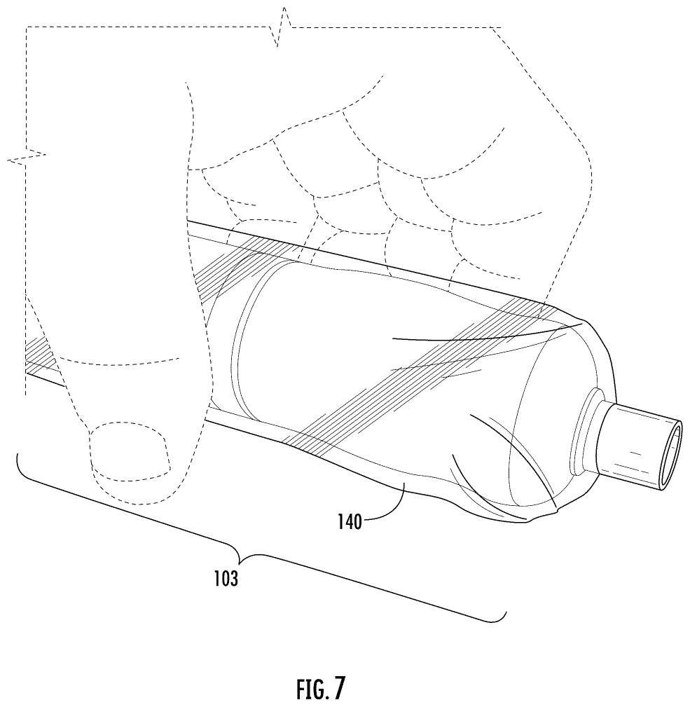

Referring now also to FIG. 5, an embodiment of a disposable elastic membrane barrier 103 is shown. In this embodiment, the disposable elastic membrane barrier 103 may include a notch 142, a pair of shoulder portions 144 (e.g., square-shaped shoulders), an integrated bag portion 140, and a seal 146. A grip 102 and an attached tattoo machine 120 may be positioned into the opening 148 of the bag portion 140 until a portion of the grip 102 and/or a needle cartridge secured to the grip 102 protrudes out of the notch 142 portion of the barrier 103 (similar to what is shown in FIG. 7). The shoulders 144 of the barrier 130 may be utilized to prevent the grip 102 and attached tattoo machine 120 from falling out of the barrier 103 when the grip 102 and tattoo machine 120 are within the barrier 103. The tattoo artist may optionally choose the seal the bag portion 140 by utilizing the seal 146. Once the barrier 103 is used, such as during a tattoo session, the tattoo machine 120 and/or grip 102 may be removed from the barrier 103 and a new barrier 103 may be used with the tattoo machine 120 and/or grip 102. Referring now also to FIG. 6, another embodiment of the disposable elastic membrane barrier 103 is shown. This embodiment may be similar to the embodiment of FIG. 5, however, instead of having squared-off shoulders 144, the barrier 103 in this embodiment may have shoulders 154 that taper and angle towards a notch 152. The tapered and angled shoulders 154 may be suitable to fit a grip 102 that has an angled and/or tapered shaped. Notably, any shape may be utilized for the shoulders 144, 154 depending on the type and/or shape of grip 102, the type and/or shape of a needle cartridge affixed to the grip 102, and/or the type and/or shape of a tattoo machine 120 attached to the grip 102. As with other barriers 103 described herein, this embodiment may also include a seal 156 that may be utilized to seal the bag portion 150 of the barrier 103 closed when the tattoo machine 120 is housed within the bag portion 150.

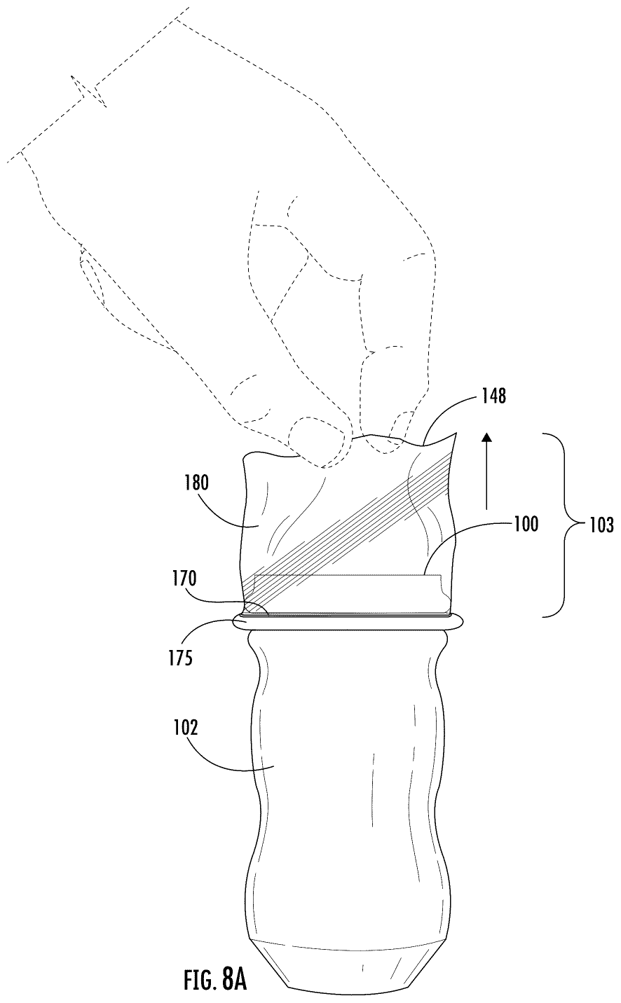

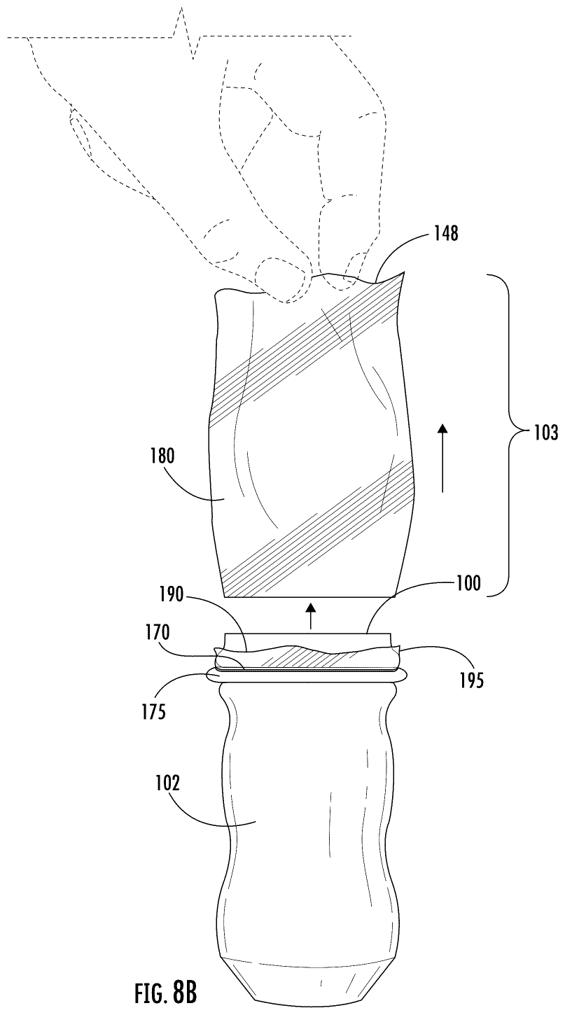

Referring now also to FIGS. 8A and 8B, a further embodiment of a disposable elastic membrane barrier 103 is shown. In this embodiment, the barrier 103 may include a ring (or other shape) portion 175 (i.e. a securing portion) and a bag portion 180. In certain embodiments, the ring portion 175 may be configured to fit and secure into a groove 125 of the grip 102, however, in certain embodiments, the ring portion 175 may be sized and shaped so that the ring portion 175 is configured to simply slide over to a selected portion of the grip 102 and stay in position once the ring portion 175 is located at the selected portion of the grip 102 (e.g., such as via a snug or friction fit). The ring portion 175 may include a groove 170, which may be configured to store any number of bag portions 180. Once the ring portion 175 is secured to the grip 102, a user (e.g., tattoo artist) may grab onto a portion of one bag portion 180 that is protruding out of the groove 170 of the ring portion 175 and pull the bag portion upwards and away from the ring portion 175, as shown in FIG. 8A. The user may continue pulling the bag portion 180 until it is a desired length. The user may then insert the tattoo machine 120 into the opening of the bag portion 180 and secure the tattoo machine 120 to the grip 102 and begin using the tattoo machine 120 during a tattoo session with a tattoo recipient. Once the user is done using the tattoo machine 120 and/or at other desired times, the user may detach the bag portion as shown in FIG. 8B. For example, the user may detach the bag portion 180 via a perforated section 190 connecting the currently used bag portion 180 to a next bag portion 195 residing within the groove 170 of the ring portion 175. Other techniques for connecting the bag portions 180, 195 may also be utilized in addition to or instead of perforated sections 190. Once the user detaches the bag portion 180 that has been used, the user may pull on the next bag portion 195 when the user wants to cover the tattoo machine 120 again for a next tattoo session. The user can keep pulling bag portions 180, 190 out of the groove 170 until the supply runs out. The user may restock additional bag portions 180, 190 into the groove 170 or may be obtain a new barrier 103 altogether that is preloaded with bag portions 180, 190.

As indicated above, the barriers 103 may be utilized to cover tattoo machines 120, which may be utilized by a tattoo artist to deliver ink to the skin of a user, such as during a tattoo session with a tattoo recipient. The tattoo machine 120 may include any number of components including, but not limited to, processors, memories, transceivers, communication devices, batteries, battery charger ports, power delivery ports, motors, needle cartridges, needles, battery packs, grips 102, accessories, or a combination thereof. In certain embodiments, the tattoo machine 120 may be configured to reciprocate the needle 101 when a tattoo artist has activated the tattoo machine 120 and power is delivered to the motor of the tattoo machine 120. The tattoo machine 120 may be configured to pair with any device, such as via cellular connection, short-range wireless connection (e.g., Bluetooth, Zigbee, Z-wave, etc.), long-range wireless connection, transceiver, physical connection, or a combination thereof. The tattoo machine 120 may also communicate with a software application executing on a device, exchange information with the software application, and receive control instructions from the software application to control the operative functionality of the tattoo machine 120. In certain embodiments, the tattoo machines 120 may include controls 122 to activate or deactivate the tattoo machine 120, adjust voltage settings of the tattoo machines, control any settings of the tattoo machine 120, or a combination thereof.

In certain embodiments, the tattoo machine 120, grip 102, and/or the barrier 103 may include sensors that may be configured to detect the presence of contaminants, detect temperature, detect when a bag portion and/or barrier 103 needs to be replaced, detect when the tattoo machine 120 is activated and/or in use, detect an age of a bag portion and/or barrier 103, and/or detect any conditions associated with the barriers 103 and/or bag portions. Based on the information obtained from the sensors, the information may be provided to the tattoo machine 120 and/or to communicatively linked devices. In certain embodiments, the barriers 103 may be fitted with processors, memories, communication devices, and/or other components to facilitate the transmission of sensor data to the tattoo machine 120 and/or devices communicatively linked to the barriers 103. In certain embodiments, the sensor data may trigger ordering of new barriers 103 for delivery to the tattoo artist, may cause an notification to be provided by the tattoo machine 120 and/or barrier 103 that the barrier 103 is contaminated and/or needs to be replaced, and/or provide any other alerts. Notably, any of the features of the embodiments of the barriers 103 described herein may be combined with each other and/or the components of the computer system 1100.

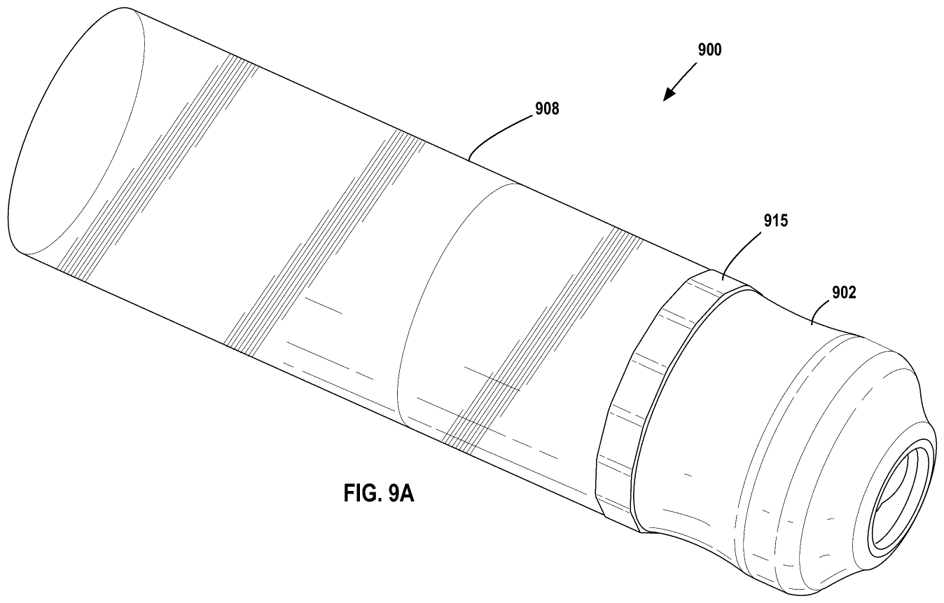

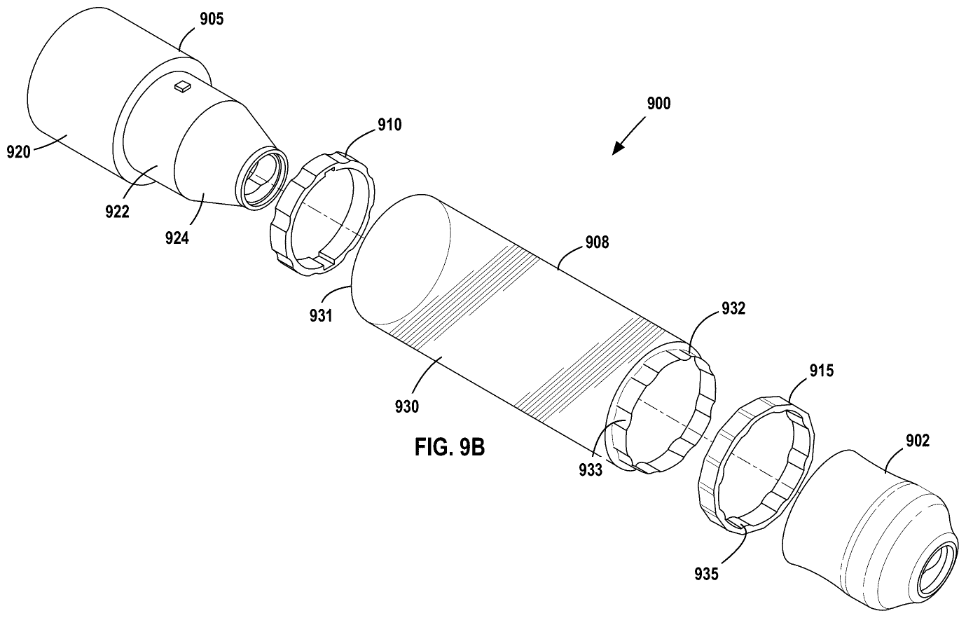

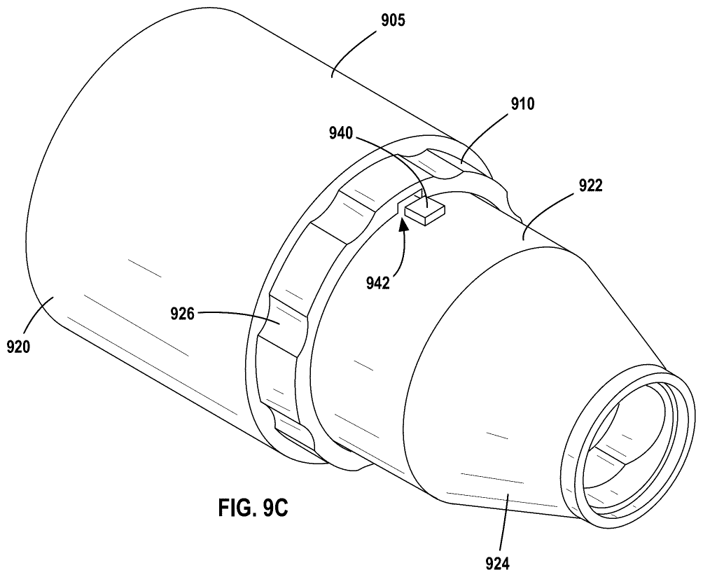

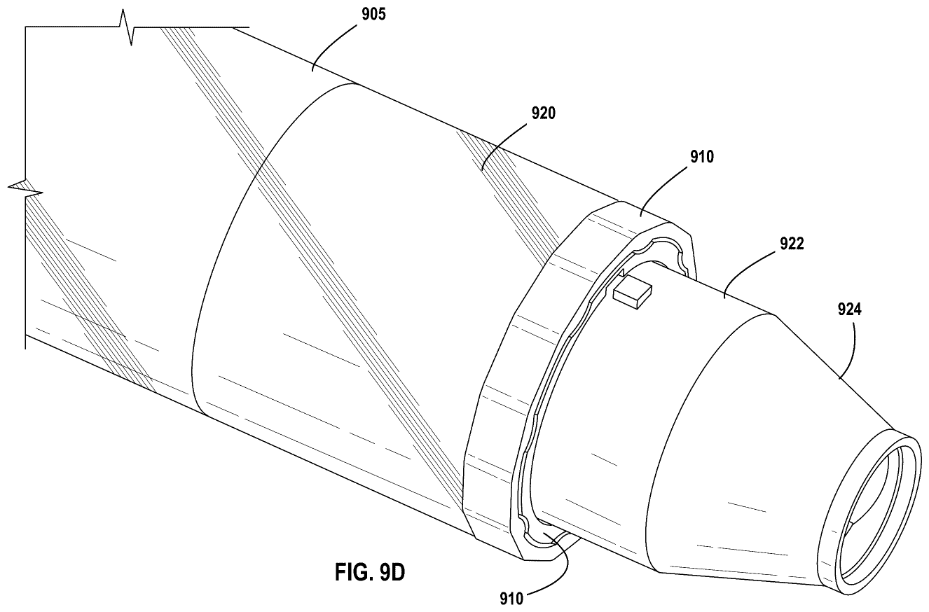

FIGS. 9A-9D illustrate an example grip assembly 900 with a rotatable sleeve 908 that can be coupled to any of the tattoo machines described herein in the various manners described above and to facilitate ergonomic and sanitary use of the tattoo machines. As shown more clearly in FIG. 9B, the grip assembly 900 includes a grip body 905, an inner ring 910, a sleeve/bag portion 908, an outer ring 915, and a grip 902. Some examples of the grip body 905, the inner ring 110, and the outer ring 915 are formed from a rigid material such as a thermoplastic material, a metal material, etc. Some examples of the grip 902 are formed from materials such as rubber, latex fabric, silicone, plastics (e.g., ABS plastic, polyethylene plastic, etc.), metals, or any combination thereof. Some examples of the sleeve 908 are formed from an elastomeric material. Some examples of the elastomeric material are rigid, whereas other examples are flexible. Examples of the material include but are not limited to plastic, rubber, elastic membrane material, stretchable materials, fabrics, any type of materials, or a combination thereof. In some examples, the volume of the sleeve 908 is sized to cover the tattoo machine 120 and/or other componentry that is affixed to the grip 102.

Some examples of the grip body 905 have a first cylindrically shaped section 920, a second cylindrically section 922 that extends from the first section, and a third generally conical-shaped section 924 that extends from the second section 922. In some examples, the inside diameter of the inner ring 910 is sized to correspond to the outside diameter of the second section 922 to facilitate sliding the inner ring 910 over the second section 922. As shown more clearly in FIG. 9C, some examples of the second section 922 include a retaining key 940 that is configured to fit within a corresponding notch 942 defined in the inner ring 910 to facilitate holding the inner ring 910 in place on the second section 922 of the grip body 905. For example, the inner ring 910 can be slid past the retaining key 940 and rotated to an extent to prevent the inner ring 910 from inadvertently falling off of the grip body 905 during the assembly process. While the example shown only illustrates a single retaining key 940 and notch 942, other examples of the grip body 905 may include multiple retaining keys and notches. In some examples, the outer diameter of the inner ring 910 is sized to be smaller than the outer diameter of the first section 920 of the grip body 905 to facilitate sliding the sleeve 908 over the inner ring 910. In some examples, the outward-facing surface of the inner ring 910 includes a series of grooves 926 distributed about the periphery of the inner ring 910.

As shown more clearly in FIG. 9B, some examples of the sleeve 908 have a generally cylindrical shaped body section 930 with a first/open end 931 having a diameter sized to facilitate sliding the sleeve 908 over the inner ring 910 and the grip body 905, and a second/securing end 932 opposite the first end 931. In some examples, the securing end 932 of the sleeve 908 defines a series of features 933 and the inside facing surfaces of these features 933 are configured to slide over and fit within the grooves 926 of the inner ring 910. Some examples of the outer ring 915 define a series of inward-facing protrusions 935 distributed about the periphery of the outer ring 915 that are configured to slide over and fit within outside facing surfaces of the features 933 of the securing end 932 of the sleeve 908 such that when assembled, the securing end 932 is sandwiched between the inner ring 910 and the outer ring 915, as shown in more clearly in FIG. 9D. In some examples, the sleeve 908 is fixed/adhered to the inner ring 910 and/or the outer ring 915 after these components are brought into alignment.

An example assembly operation of the grip assembly 900 of FIGS. 9A-9D involves aligning the notch 942 of the inner ring 910 with the retaining key 940 of the second section 922 of the grip body 950 and then sliding the inner ring 910 over the grip body 905 until the inner ring 910 passes the retaining key 940. Next, the inner ring 910 is rotated about the second section 922 so that the notch 942 and the retaining key 940 are no longer in alignment. This, in turn, prevents the inner ring 910 from sliding passed the retaining key 940. The first end 931 of the sleeve 908 is then slid over the inner ring 910 and the grip body 905 until the securing end 932 of the sleeve 908 slides over the inner ring 910. The outer ring 915 is then slid over the securing end 932 of the sleeve 908. In some examples, the grip 902 is then formed over the third section 924 and at least a portion of the second section 922 of the grip body 950 to complete the assembly. In some examples, the grip 902 is provided as a separate component, which is then slid over the third section 924 and at least part of the second section 922 of the grip body and adhered to the third section 924 and/or the second section 922 to complete the assembly. After assembly, the sleeve 908 can be rotated freely about the second section of the grip body 905.

Although FIGS. 1-9D illustrates specific example configurations of disposable elastic membrane barrier, grips 102, and the tattoo machine 120, these devices may include any configuration of the components, which may include using a greater or lesser number of the components. For example, the disposable elastic membrane barriers 103, grips 102, tattoo machine 120, and/or any of the other components shown in FIGS. 1-9D may include any number of grips, barriers 103, bag portions (e.g., bag portions 108, 112, 114, 134, 140, 150, 180), securing portions (e.g., 106, 132, 170), and/or other components. Additionally, the disposable elastic membrane barrier may serve as an accessory to the tattoo machine 120. In certain embodiments, the tattoo machine 120 and/or grip 102 may include communication devices (e.g., wireless chips, transceivers, etc.), which may communicatively link with and transmit and receive data from any other device, such as, but not limited to, a mobile device (e.g., smartphone, smartwatch, smart device, computer, laptop, etc.), a footswitch, a hover device, any type of accessory, any type of device, or a combination thereof.

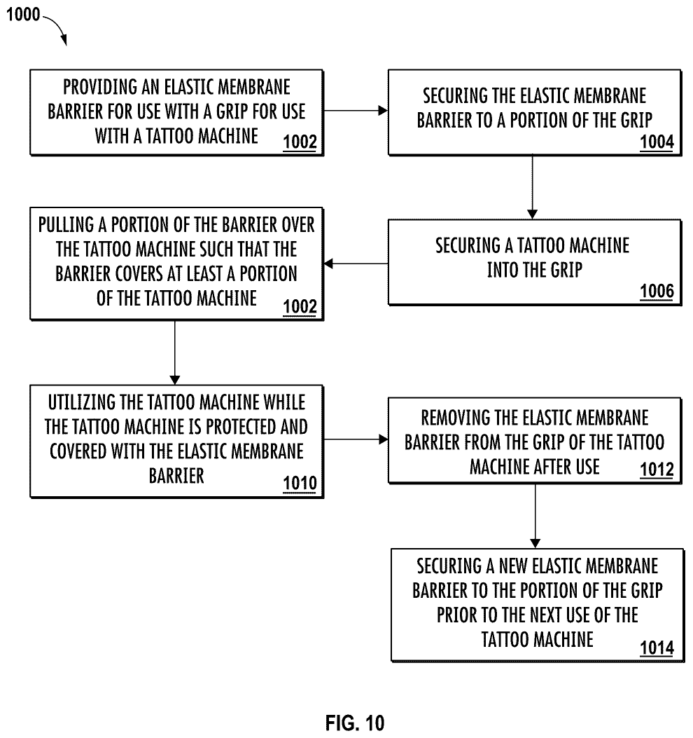

Notably, as shown in FIG. 10, an exemplary method 1000 for utilizing a grip with a disposable barrier for use with a tattoo machine is schematically illustrated. The method 1000 may include steps for securing a disposable barrier (e.g., disposable barrier 140, 150, and the like) to a grip (e.g., grip 102) to facilitate protection of a tattoo machine 120 connected to the grip and reduce potential contamination as well. The method 1000 may include, at step 1002, providing a disposable elastic membrane barrier (e.g., barrier 103, 140, 150, etc.) for use with a grip utilized with a tattoo machine (e.g., tattoo machine 120). The elastic membrane barrier, when deployed, may be utilized to cover a portion of the grip and some or all of a tattoo machine attached to the grip. When deployed, the elastic membrane barrier may shield the tattoo machine and other componentry from disease vectors, pathogens, bacteria, chemicals, ink, other contaminants, or a combination thereof. In certain embodiments, the providing of the barrier may be facilitated by a user or potentially a dispenser utilized to store one or more barriers. At step 1004, the method 1000 may include securing the elastic membrane barrier to a portion of the grip. For example, a securing portion of the elastic membrane barrier may be glued or otherwise adhered to the portion of the grip. In certain embodiments, the securing portion of the elastic membrane barrier may be a ring-shaped component (or other desired shape) that may be configured to lock or otherwise fit into place within a groove located on the portion of the grip. In certain embodiments, the securing portion of the elastic membrane barrier may be configured to secure within an overmolded portion of the grip such that the securing portion is hidden away from direct view of a tattoo machine user. Of course, other techniques for securing the securing portion of the elastic membrane barrier may also be utilized as well. In certain embodiments, the elastic membrane barrier may include a barrier bag portion that may be configured to cover a tattoo machine, a portion of the grip, and/or other tattoo machine componentry when extended away from the securing portion of the elastic membrane barrier. When not in use or at other desired times, the barrier bag portion may be collapsed back towards the securing portion, thereby revealing the tattoo machine and/or other componentry previously covered by the barrier bag portion. In certain embodiments, the securing of the elastic membrane barrier may be performed by a user (e.g., tattoo artist), a machine, or a combination thereof.

At step 1006, the method 1000 may include securing a tattoo machine onto the grip. For example, the tattoo machine may be locked into place onto a receptacle of the grip so that the grip may be held by a tattoo artist while utilizing the tattoo machine/grip combination. In certain embodiments, the securing of the tattoo machine onto the grip may be performed by a user, a machine, or a combination thereof. At step 1008, the method 1000 may include pulling, extending, or otherwise deploying the barrier bag portion away from the securing portion of the clastic membrane barrier to cover a portion of the grip, the connected tattoo machine, and/or any other tattoo machine componentry and/or accessories. In certain embodiments, the pulling, extending, or otherwise deploying of the barrier bag may be performed by a user, a machine, or a combination thereof. Once the componentry is covered by the barrier bag of the clastic membrane barrier, the method 1000 may include, at step 1010, utilizing the tattoo machine while the tattoo machine is protected and covered by the elastic membrane barrier. For example, a tattoo machine artist may hold onto the grip and proceed to apply a tattoo on a tattoo recipient during a tattooing session. In certain embodiments, the utilizing of the tattoo machine may be performed by a user, a machine, or a combination thereof.

Once the tattoo machine is no longer being used or at any other desired time, the method 1000 may proceed to step 1012. At step 1012, the method 1000 may include removing the clastic membrane barrier from the grip of the tattoo machine. In certain embodiments, instead of removing the entire clastic membrane barrier, only the barrier bag portion may be removed and detached from the securing portion. In certain embodiments, the removing of the clastic membrane barrier may be performed by a user, a machine, or a combination thereof. At step 1014, the method 1000 may include securing a new elastic membrane barrier to the portion of the grip prior to the next use of the tattoo machine. In certain embodiments, if only the barrier bag portion was removed in step 1012, the method 1000 may include attaching or otherwise affixing a new barrier bag to the previous securing portion of the elastic membrane barrier. Once the new elastic membrane barrier (or barrier bag portion) is secured to the grip, the tattoo machine be utilized again. The method 1000 may be repeated as necessary for each tattoo machine use or at other desired times. Notably, the method 1000 may further incorporate any of the features and functionality described herein.

Referring now also to FIG. 11, at least a portion of the methodologies and techniques described with respect to the exemplary embodiments of the grip 102 and/or tattoo machine 120 can incorporate a machine, such as, but not limited to, computer system 1100, or other computing device within which a set of instructions, when executed, may cause the machine to perform any one or more of the methodologies or functions discussed above. The machine may be configured to facilitate various operations conducted by the tattoo machine 120. For example, the machine may be configured to, but is not limited to, assist the tattoo machine 120 by providing processing power to assist with processing loads experienced in the tattoo machine 120, by providing storage capacity for storing instructions or data traversing the tattoo machine 120, or by assisting with any other operations conducted by or within the tattoo machine 120. As another example, the computer system 1100 may assist with adjusting settings of the tattoo machine 120, regulating the amount of power delivered to the motor of the tattoo machine 120, activating or deactivating the tattoo machine 120, alternating between power sources that are utilized for powering the tattoo machine 120, and/or performing any operation described herein.

In some embodiments, the machine may operate as a standalone device. In some embodiments, the machine may be connected (e.g., using a communications network 1135, another network, or a combination thereof) to and assist with operations performed by other machines and systems, such as, but not limited to, the tattoo machine 115, any other system, program, and/or device, or any combination thereof. The machine may be connected with any component of the grip 102, the tattoo machine 120, or a combination thereof. In certain embodiments, the machine may reside within the grip 102 and/or tattoo machine 120. In a networked deployment, the machine may operate in the capacity of a server or a client user machine in a server-client user network environment, or as a peer machine in a peer-to-peer (or distributed) network environment. The machine may comprise a server computer, a client user computer, a personal computer (PC), a tablet PC, a laptop computer, a desktop computer, a control system, a network router, switch or bridge, or any machine capable of executing a set of instructions (sequential or otherwise) that specify actions to be taken by that machine. Further, while a single machine is illustrated, the term “machine” shall also be taken to include any collection of machines that individually or jointly execute a set (or multiple sets) of instructions to perform any one or more of the methodologies discussed herein.

The computer system 1100 may include a processor 1102 (e.g., a central processing unit (CPU), a graphics processing unit (GPU, or both), a main memory 1104 and a static memory 1106, which communicate with each other via a bus 1108. The computer system 1100 may further include a video display unit 1110, which may be, but is not limited to, a liquid crystal display (LCD), a flat panel, a solid state display, or a cathode ray tube (CRT). The computer system 1100 may include an input device 1112, such as, but not limited to, a keyboard, a cursor control device 1114, such as, but not limited to, a mouse, a disk drive unit 1116, a signal generation device 1118, such as, but not limited to, a speaker or remote control, and a network interface device 1120.

The disk drive unit 1116 may include a machine-readable medium 1122 on which is stored one or more sets of instructions 1124, such as, but not limited to, software embodying any one or more of the methodologies or functions described herein, including those methods illustrated above. The instructions 1124 may also reside, completely or at least partially, within the main memory 1104, the static memory 1106, or within the processor 1102, or a combination thereof, during execution thereof by the computer system 1100. The main memory 1104 and the processor 1102 also may constitute machine-readable media.

In certain embodiments, the machine may also include any of the components of the grip 102, the tattoo machine 120, and/or other components as described herein. The machine may be configured to reside within any of the components of the grip 102, the tattoo machine 120, both the grip 102 and tattoo machine 120, as a standalone machine, and/or in other devices.

Dedicated hardware implementations including, but not limited to, application specific integrated circuits, programmable logic arrays and other hardware devices can likewise be constructed to implement the methods described herein. Applications that may include the apparatus and systems of various embodiments broadly include a variety of electronic and computer systems. Some embodiments implement functions in two or more specific interconnected hardware devices or devices with related control and data signals communicated between and through the devices, or as portions of an application-specific integrated circuit. Thus, the example system is applicable to software, firmware, and hardware implementations.

In accordance with various embodiments of the present disclosure, the methods described herein are intended for operation as software programs running on a computer processor. Furthermore, software implementations can include, but not limited to, distributed processing or component/object distributed processing, parallel processing, or virtual machine processing can also be constructed to implement the methods described herein.

The present disclosure contemplates a machine-readable medium 1122 containing instructions 1124 so that a device connected to the communications network 1135, another network, or a combination thereof, can send or receive voice, video or data, and communicate over the communications network 1135, another network, or a combination thereof, using the instructions. The instructions 1124 may further be transmitted or received over the communications network 1135, another network, or a combination thereof, via the network interface device 1120.

While the machine-readable medium 1122 is shown in an example embodiment to be a single medium, the term “machine-readable medium” should be taken to include a single medium or multiple media (e.g., a centralized or distributed database, and/or associated caches and servers) that store the one or more sets of instructions. The term “machine-readable medium” shall also be taken to include any medium that is capable of storing, encoding or carrying a set of instructions for execution by the machine and that causes the machine to perform any one or more of the methodologies of the present disclosure.

The terms “machine-readable medium,” “machine-readable device,” or “computer-readable device” shall accordingly be taken to include, but not be limited to: memory devices, solid-state memories such as a memory card or other package that houses one or more read-only (non-volatile) memories, random access memories, or other re-writable (volatile) memories; magneto-optical or optical medium such as a disk or tape; or other self-contained information archive or set of archives is considered a distribution medium equivalent to a tangible storage medium. The “machine-readable medium,” “machine-readable device,” or “computer-readable device” may be non-transitory, and, in certain embodiments, may not include a wave or signal per se. Accordingly, the disclosure is considered to include any one or more of a machine-readable medium or a distribution medium, as listed herein and including art-recognized equivalents and successor media, in which the software implementations herein are stored.

The illustrations of arrangements described herein are intended to provide a general understanding of the structure of various embodiments, and they are not intended to serve as a complete description of all the elements and features of apparatus and systems that might make use of the structures described herein. Other arrangements may be utilized and derived therefrom, such that structural and logical substitutions and changes may be made without departing from the scope of this disclosure. Figures are also merely representational and may not be drawn to scale. Certain proportions thereof may be exaggerated, while others may be minimized. Accordingly, the specification and drawings are to be regarded in an illustrative rather than a restrictive sense.

Thus, although specific arrangements have been illustrated and described herein, it should be appreciated that any arrangement calculated to achieve the same purpose may be substituted for the specific arrangement shown. This disclosure is intended to cover any and all adaptations or variations of various embodiments and arrangements of the invention. Combinations of the above arrangements, and other arrangements not specifically described herein, will be apparent to those of skill in the art upon reviewing the above description. Therefore, it is intended that the disclosure not be limited to the particular arrangement(s) disclosed as the best mode contemplated for carrying out this invention, but that the invention will include all embodiments and arrangements falling within the scope of the appended claims.

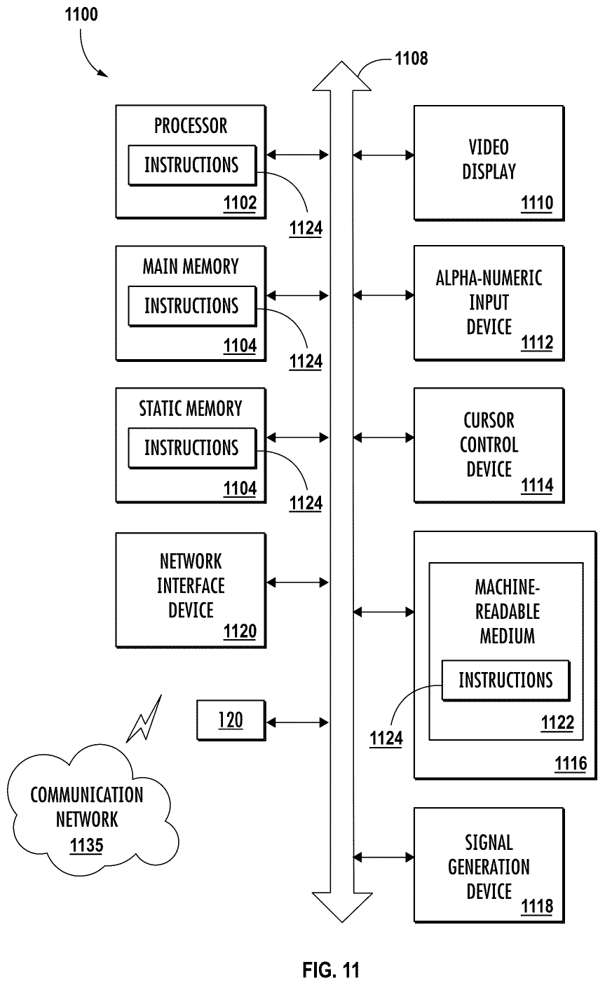

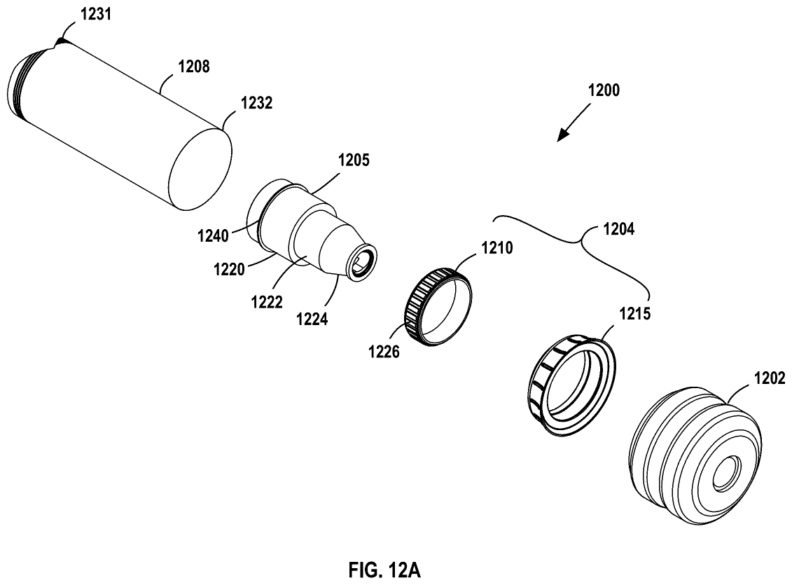

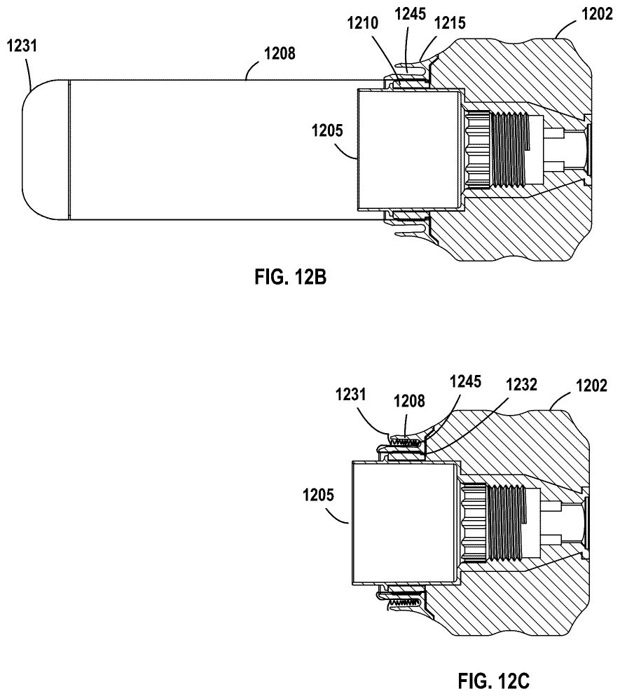

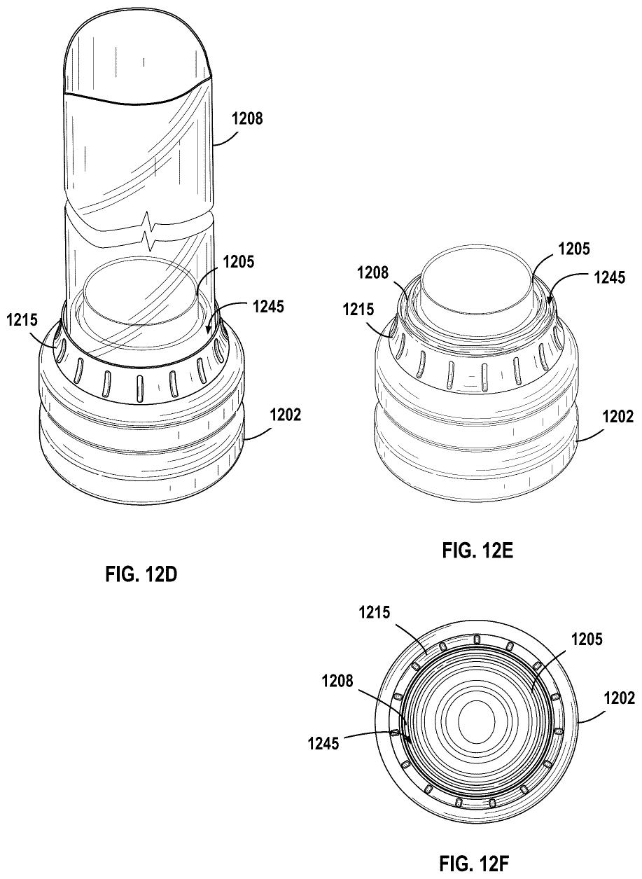

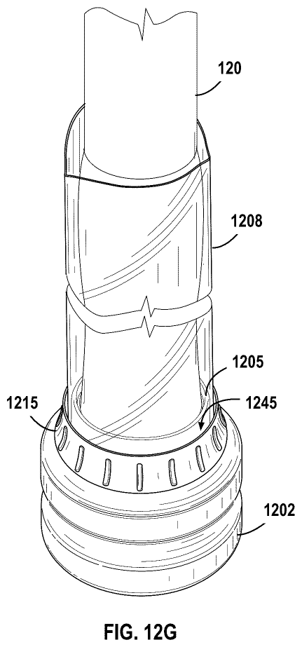

FIGS. 12A-12G illustrate an example grip assembly 1200 with a rotatable sleeve or barrier or bag 1208 that can be coupled to any of the tattoo machines described herein in the various manners described above and to facilitate ergonomic and sanitary use of the tattoo machines. As shown more clearly in FIG. 12A, the grip assembly 1200 includes a grip body 1205, a securing portion 1204 that comprises an inner ring 1210 and an outer ring 1215, a sleeve/bag portion 1208, and a grip 1202. Some examples of the grip body 1205, the inner ring 1210, and the outer ring 1215 are formed from a rigid material such as a thermoplastic material, metal material, etc. Some examples of the grip 1202 are formed from materials such as rubber, latex fabric, silicone, plastics (e.g., ABS plastic, polyethylene plastic, etc.), metals, or any combination thereof. Some examples of the sleeve 1208 are formed from an elastomeric material. Some examples of the elastomeric material are rigid, whereas other examples are flexible. Examples of the material include but are not limited to plastic, rubber, elastic membrane material, stretchable materials, fabrics, any type of materials, or a combination thereof. In some examples, the volume of the sleeve 1208 is sized to cover the tattoo machine 120 and/or other componentry that is affixed to the grip 102.

Some examples of the grip body 1205 have a first cylindrically shaped section 1220 having a first diameter, a second cylindrically section 1222 having a second diameter smaller than the first diameter and that extends from the first section and a third generally conical-shaped section 1224 that extends from the second section 1222 and tapers to a third diameter that is smaller than the second diameter. In some examples, the inside diameter of the inner ring 1210 of the securing portion 1204 is sized to correspond to the first diameter to facilitate sliding the inner ring 1210 over the first cylindrically shaped section 1220 and rotating the inner ring 1210, and therefore, the securing portion 1204, about the first cylindrically shaped section 1220. In this regard, some examples of the first cylindrically shaped section 1220 include a ledge or stop 1240 that prevents the inner ring 1210 from sliding past the end of the first cylindrically shaped section 1220.

As shown in FIGS. 12B and 12C, some examples of the sleeve 1208 have a generally cylindrical-shaped body with a diameter sized to facilitate sliding the sleeve 1208 over the inner ring 1210 and the grip body 1205. When assembled, a first end 1232 of sleeve 1208 is positioned over the inner ring 120. In such an arrnagemnet, the components can be coeccentric.

The outer ring 1215 of the securing portion 1204 has an inner diameter sized to facilitate sliding the outer ring 1215 over the first end 1232 of the sleeve 1208 such that when assembled, the first end 1232 of the sleeve 1208 is sandwiched, secured or friction-fit between the inner ring 1210 and the outer ring 1215. In this regard, some examples of the outward-facing surface of the inner ring 1210 include a series of compressible grooves or bumps 1226 distributed about the periphery of the inner ring 1210. These bumps 1226 resiliently compress when the outer ring 1215 is slid over the inner ring 1210 and provide a compressive force between the inner ring 1210 and the outer ring 1215 that secures the inner ring 1210 and the outer ring 1215 to one another.

As shown in FIGS. 12B and 12C, some examples of the outer ring 1215 define an annular space 1245. As shown in FIG. 12C, in some examples, after the sleeve 1208 is sandwiched between the inner ring 1210 and the outer ring 1215, the sleeve 1208 is scrunched, collapsed in an accordian manner, or otherwise compressed in a stored or non-deployed state, within the annular space 1245 leaving a small portion of the second end 1231 of the sleeve 1208 exposed. Thus, the annular space 1245 can be a storage recess or compartment for storing the sleave 1208 prior to be released, deployed or unraveled over tatoo device. Providing the annular space 1245 in the securing portion 1204 and scrunching, compressing or fitting the sleeve 1208 within the annular space 1224 facilitates providing the grip assembly 1200 in a more compact form and ready to be deployed, unraveled after a tattoo machine is inserted. After the grip assembly 1220 is coupled to a tattoo machine, the exposed second end 1231 of the sleeve 1208 may be tugged on to pull the sleeve 1208 out of the annular space 1245 of the securing portion 1204 and over the tattoo machine.

An example assembly operation of the grip assembly 1200 of FIGS. 12A-12G involves sliding the inner ring 1210 over the grip body 1205 and up to the ledge 1240 of the grip body 1205. Next, the second end 1231 of the sleeve 1208 is slid over and past the inner ring 1210 of the securing portion 1204 and the grip body 1205 until the first end 1232 of the sleeve 1208 is positioned over the inner ring 1210. The outer ring 1215 is then slid over the first end 1232 of the sleeve 1208 and the inner ring 1210 to sandwich the first end 1232 of the sleeve 1208 between the inner ring 1210 and the outer ring 1215 and, therefore, secure the sleeve 1208 within the securing portion 1204. Such securement can be without adhesives, bonding or chemical adherement. In other arrangements, adhesives, bonding or chemical adherement can be used to adhere at least a portion of the sleave 1208 to the inner ring 1210 and/or the outer ring 1215.

The grip 1202 is then positioned over the third section 1224, the second section 1222, and at least a portion of the first section 1222 of the grip body 1205 to complete the assembly. Afterward, the grip 1202 prevents the securing portion 1204 from being slid off the grip body 1205. In this regard, in some examples, the grip 1202 is adhered to one or more of the sections of the grip body 1205 to fix the grip 1202 to the grip body 1205. After assembly, the grip assembly 1200 may be coupled to a tattoo machine. In operation, after coupling the tattoo machine to the grip assembly, a user may hold the grip 1202 of the grip assembly 1200 with one hand and tug on the exposed second end 1231 of the sleeve 1208 with the other hand to pull the sleeve 1208 out of the annular space 1245. The user may also rotate the securing section 1204 with the other hand to rotate the sleeve 1208 about the longitudinal axis of the tattoo machine as needed. Additionally, the grip 1202 can freely rotate without rotating the securing portion 1204 or the sleeve 1208. Thus, the grip 1202 and/or tattoo machine can rotate indepent of the securing portion 1204 and the sleeve 1208. This allows the sleeve 1208 to remain staitionary while the grip 1202 is rotated.

The foregoing is provided for purposes of illustrating, explaining, and describing embodiments of this invention. Modifications and adaptations to these embodiments will be apparent to those skilled in the art and may be made without departing from the scope or spirit of this invention. Upon reviewing the aforementioned embodiments, it would be evident to an artisan with ordinary skill in the art that said embodiments can be modified, reduced, or enhanced without departing from the scope and spirit of the claims described below.

We claim:

1. A disposable barrier for use with a grip, comprising:
a securing portion configured to secure the disposable barrier to a portion of the grip; and
a barrier bag portion attached to the securing portion,
wherein the barrier bag portion is configured to extend away from the securing portion to cover a portion of the grip, a tattoo machine secured to the grip, or a combination thereof, when the disposable barrier is in an open configuration, and
wherein the barrier bag portion is configured to collapse towards the securing portion when the disposable barrier is a closed configuration.

2. The disposable barrier of claim 1, wherein the securing portion of the disposable barrier is configured to prevent ink back flow from a cartridge needle attached to the grip from entering the tattoo machine.

3. The disposable barrier of claim 1, wherein the securing portion comprises a ring configured to secure to a groove positioned on the grip and extending around a circumference of the grip.

4. The disposable barrier of claim 1, wherein the securing portion is configured to be glued to the portion of the grip.

5. The disposable barrier of claim 1, wherein the portion of the grip comprises an overmolded portion configured to secure to the securing portion of the disposable barrier.

6. The disposable barrier of claim 1, wherein the barrier bag portion comprises a seal positioned proximate to a first end of the barrier bag portion located opposite to a second end of the barrier bag portion attached to the securing portion of the disposable barrier.

7. The disposable barrier of claim 6, wherein the seal is configured to be sealed by a user of the disposable barrier to facilitate reduction of contamination of a tattoo machine covered by the disposable barrier.

8. The disposable barrier of claim 1, wherein the disposable barrier is configured to spin around independently of the grip, thereby enabling the grip to adjust freely while the disposable barrier is protecting the tattoo machine.

9. The disposable barrier of claim 1, wherein the disposable barrier comprises silicone, plastic, elastic material, or a combination thereof.

10. The disposable barrier of claim 1, wherein the barrier bag portion is configured to reside within a groove of the securing portion when the barrier bag portion is collapsed completely towards the securing portion.

11. The disposable barrier of claim 1, wherein the securing portion comprises an annular space within which the barrier bag portion is configured to collapse when the disposable barrier is in a closed configuration.

12. A method for utilizing a disposable barrier with a grip, comprising:
providing the disposable barrier for the grip for use with a tattoo machine configured to be attached to the grip;
securing the disposable barrier to a portion of the grip;
securing the tattoo machine to the grip;
pulling a portion of the disposable barrier over the tattoo machine such that the disposable barrier covers at least a portion of the tattoo machine;
removing the disposable barrier from the grip after the tattoo machine has been utilized; and
securing a new disposable barrier to the portion of the grip prior to a next use of the tattoo machine.

13. The method of claim 12, further comprising securing the disposable barrier to the portion of the grip by positioning a ring positioned at an end of the disposable barrier into a groove located at the portion of the grip.

14. The method of claim 12, further comprising sealing an end of the disposable barrier after pulling the portion of the disposable barrier over the tattoo machine.

15. The method of claim 12, further comprising securing the disposable barrier to the portion of the grip by adhering a portion of the disposable barrier to the portion of the grip.

16. The method of claim 12, further comprising facilitating spinning of the disposable barrier around the grip and independently of the grip, thereby enabling the grip to adjust freely while the disposable barrier is protecting the tattoo machine.

17. The method of claim 12, further comprising preventing leakage of ink from a needle cartridge of the tattoo machine into the tattoo machine by utilizing the disposable barrier.

18. The method of claim 12, further comprising utilizing the tattoo machine after the portion of the disposable barrier has been pulled over the tattoo machine.

19. A grip, comprising:
a portion configured to secure to a securing portion of a disposable barrier,
wherein the disposable barrier includes a barrier bag portion attached to the securing portion of the disposable barrier,
wherein the barrier bag portion is configured to extend away from the securing portion to cover a portion of the grip, a tattoo machine secured to the grip, or a combination thereof, and
wherein the barrier bag portion is configured to collapse towards the securing portion to uncover the portion of the grip, the tattoo machine secured to the grip, or a combination thereof; and
wherein the barrier bag is arranged with the securing portion such that the grip rotates independently of the barrier bag.

20. The grip of claim 19, wherein the grip further comprises a groove extending around a portion of the grip and configured to receive the securing portion of the disposable barrier.

21. The grip of claim 19, wherein the grip further comprises a receptable for receiving a needle cartridge of the tattoo machine and a different receptacle for securing the grip to the tattoo machine.
